(19) United States
(12) Patent Application Publication
(10) Pub. No.: US 2013/0066116 A1
(43) Pub. Date: Mar. 14, 2013

(54) METHOD FOR THE DEPOLYMERIZATION OF LIGNOCELLULOSIC BIOMASS

(71) Applicants: Frédéric Goettmann, Les Angles (FR); Philippe Makowski, Bagnols sur Ceze (FR); Denilson Da Silva Perez, Pontcharra Sur Breda (FR); Michel Petit-Conil, Grenoble (FR)

(72) Inventors: Frédéric Goettmann, Les Angles (FR); Philippe Makowski, Bagnols sur Ceze (FR); Denilson Da Silva Perez, Pontcharra Sur Breda (FR); Michel Petit-Conil, Grenoble (FR)

(73) Assignee: INSTITUT TECHNOLOGIQUE FCBA, Paris (FR)

(21) Appl. No.: 13/635,157

(22) PCT Filed: Mar. 17, 2011
(86) PCT No.: PCT/FR11/50537

§ 371 Date: Nov. 26, 2012

(30) Foreign Application Priority Data
Mar. 18, 2010 (FR) 1051928

(51) Int. Cl. C07C 41/18; C07C 43/23

(52) U.S. Cl. 568/652

(57) ABSTRACT

The present invention relates to a method for the depolymerization of lignin or of derivatives thereof, including a step of heating the lignin or the derivatives thereof in the presence of a hydroxide of general formula M(OH)n or of a mixture of M(OH)n hydroxides, where M is a metal of the alkali or alkaline-earth family and n is equal to 1 or 2, and where the mass ratio between said hydroxide or mixture of hydroxides and the lignin or the derivatives thereof is comprised between around 0.5 and around 20.

### PRIOR ART

The present invention relates to the technical field of the production of organic molecules from lignin or from lignocellulosic biomass. More precisely, the invention relates to a novel method of depolymerization/degradation of products containing lignin under ionothermal conditions.

With the expected increasing scarcity of easily accessible fossil resources, the petroleum and chemical industries are turning increasingly to biomass as a source of bound carbon for the synthesis of the molecules that they need.

In the field of energy, the term biomass includes all organic materials that can be used as energy sources. The latter are essentially derived from plants.

Biomass is mainly constituted by carbohydrate biomass such as cereals, sugar beet or sugar cane, oleaginous biomass such as colza or oil palm, and lignocellulosic biomass comprising, amongst others, wood, green residues in general or straw.

The products extracted from lignocellulosic biomass contain, amongst other things, an organic polymer called lignin.

Lignin is a phenolic macromolecule, whose structure is still poorly known. It is present in the cell walls of many plants (especially wood), to which it confers their properties of rigidity. It is available in the black liquor from pulp production (between 100 and 150 million tonnes of lignin is “produced” in this way each year), but can also be extracted directly from wood chips or from straw from annual plants. At present, the lignin extracted from the industrial production of cellulose is used for the recovery of the reagents involved in the kraft process and burned in order to ensure energy self-sufficiency of the extraction processes.

The lignins are polymers of monolignols. There are at least three different types of monomers: coumaryl alcohol, coniferyl alcohol and sinapyl alcohol.

Whereas many production processes of chemical compounds from cellulose permit the production of chemical compounds on an industrial scale, at present there is no economically viable means for producing these chemical compounds from lignocellulosic biomass.

For example, in the papermaking industry, about 50 million tonnes of lignin are produced as byproducts and waste products every year and only about 1 to 2% is recycled and reused.

There are already various industrial processes for utilizing biomass and especially lignocellulosic biomass.

One of these processes, called the bisulfite process, can be used for producing vanillin on an industrial scale via the oxidative degradation of lignosulfonic acids.

Numerous processes allowing for the degradation of lignin are known but they all have some problems limiting their use on an industrial scale, amongst other things, due to inadequate suitability to industrial requirements and/or incompatibility with increasingly strict environmental requirements.

Amongst these known methods, it may be mentioned a method described in U.S. Pat. No. 5,807,952 which uses high-temperature pyrolysis (between 400° C. and 600° C.) in solid phase or in the presence of a small amount of a basic catalyst such as potassium hydroxide.

Other methods are those described for example in U.S. Pat. No. 5,959,167 which relate to the depolymerization of lignin under solvothermal conditions, i.e. in an alcohol-diluted medium heated to a temperature near their critical point (between 250° C. and 310° C.), and therefore taking place under pressure conditions of around 140 bar (2000 psi).

The depolymerization of lignin has also been carried out in reductive conditions with, amongst others, catalysts containing transition metals such as nickel, palladium, or platinum, in a dilute aqueous medium and at a pressure of about 140 bar (2000 psi). An example of this type of method is described in U.S. Pat. No. 2,220,624. Metal complexes containing transition metals have also been described for their catalytic properties in reactions of degradation of lignin, for example in international patent application published under No. WO 2008/106811.

The methods of degradation of lignin described in the prior art are diverse and although they employ a wide range of methods and operating conditions, they nevertheless all have similar drawbacks.

The distribution of molecular weight and of chemical functionalities of the organic products obtained by degradation of lignin is generally very broad.

The mixtures obtained often contain a multitude of degradation products and/or products with very similar chemical structure, so that generally it is not easy to separate the different constituents of the mixture.

Incidentally, due to the multitude of products generated, the amount of the degradation products that can be isolated is generally not economically advantageous.

It is therefore desirable to obtain a mixture of degradation products from lignocellulosic biomass that only has a limited number of compounds, so that they can be isolated at economically viable yields.

Moreover, good conversion of lignin to organic molecules is generally only obtained using harsh experimental conditions (in terms of temperature and pressure) and therefore expensive.

It is therefore also desirable to be able to depolymerize lignin into “useful” organic molecules using milder experimental conditions, preferably under atmospheric pressure and at temperatures below 300° C., thus allowing, on the one hand, a significant reduction of costs, and on the other hand, easier industrial application of the method, for example not requiring the use of autoclaves.

It should also be noted that most of the known methods for degrading lignin employ catalysts based on transition metals, which are sensitive to poisoning, and are expensive and/or toxic.

It is therefore desirable, not only for economic reasons but also for environmental reasons, to be able to depolymerize lignin or derivatives thereof contained in lignocellulosic biomass by a method that does not use transition metals.

Accordingly, a method combining at least one of the aforementioned advantages, and preferably all of the aforementioned advantages, would be very advantageous as it would make it possible to produce organic molecules, preferably aromatic, from lignin or derivatives thereof more easily, less expensively and in a more environment-friendly manner.

### DESCRIPTION OF THE DRAWINGS

FIG. 1 represents the chromatogram obtained by GC-MS analysis of a sample of a mixture obtained by depolymerization of lignin obtained from conifers, purified according to the method of the invention.

FIG. 2 represents the chromatogram obtained by GC-MS analysis of a sample of a mixture obtained bydepolymerization of lignin obtained from the treatment of pine chips according to the method of the invention.

FIG. 3 represents the chromatogram obtained by GC-MS analysis of a sample of a mixture obtained bydepolymerization of lignin obtained from the treatment of cane from Provence according to the method of the invention.

FIG. 4 represents the chromatogram obtained by GC-MS analysis of a sample of a mixture obtained bydepolymerization of lignin obtained from the treatment of lignin from annual plants according to the method of the invention.

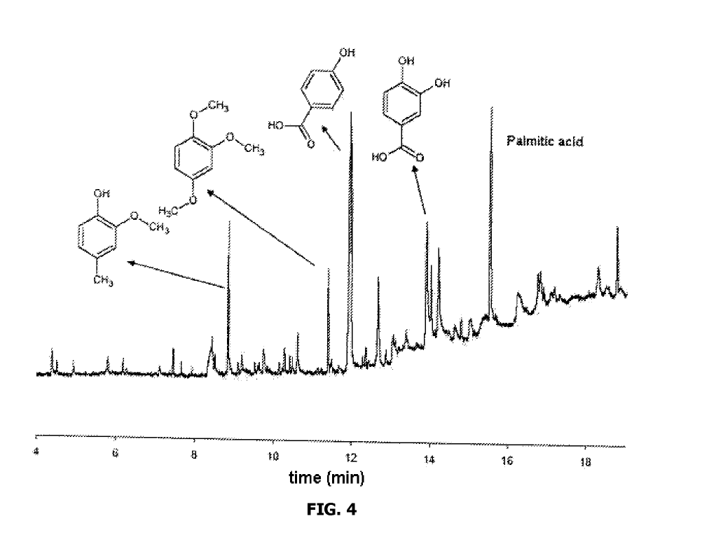

FIG. 5 represents the chromatogram obtained by GC-MS analysis of a sample of a mixture obtained bydepolymerization of lignin obtained from the treatment of a black liquor from conifers according to the method of the invention.

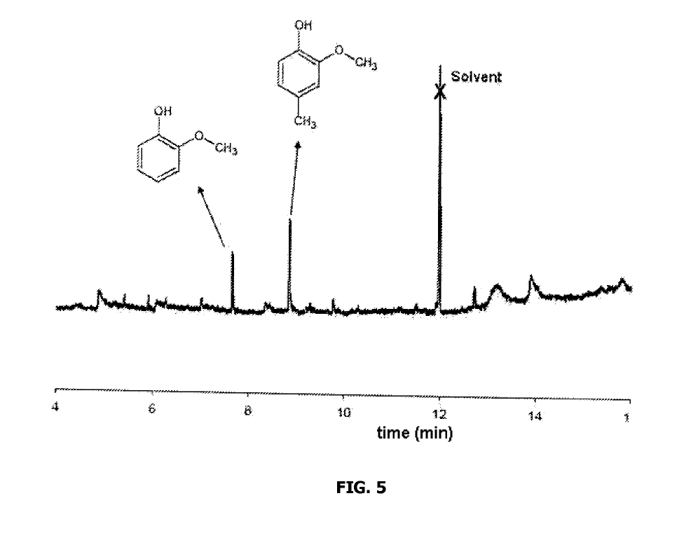

FIG. 6 represents the chromatogram obtained by GC-MS analysis of a sample of a mixture obtained bydepolymerization of lignin obtained from the treatment of a lignosulfonate according to the method of the invention.

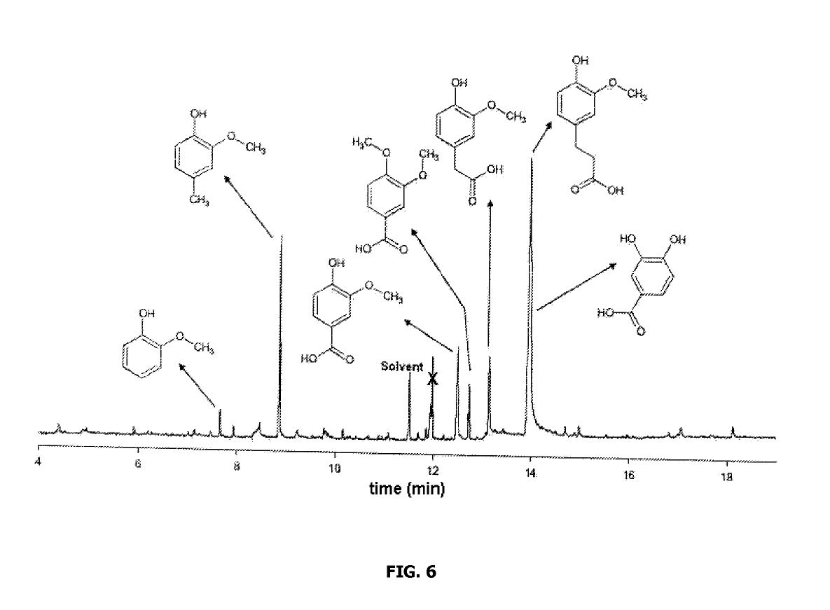

### DESCRIPTION OF THE INVENTION

The invention relates to a method for the depolymerization of lignin or of the derivatives thereof notably having the following advantages:
the use of relatively mild experimental conditions, preferably reaction temperatures below 250° C. under atmospheric pressure, notably making it possible to reduce production costs and simplify the implementation of the method,
obtaining a relatively limited number of degradation products, making it possible, among other things, to facilitate the isolation of “useful” organic molecules,
flexibility of the method in terms of the sources of substrate that can be used as starting material,
high degree of cleanness and easy purification of the raw mixture of degradation products,
good reproducibility with respect to the nature of the organic molecules produced by the method regardless of the source of lignin used,
absence of catalyst containing transition metals, which notably makes it possible to meet the environmental requirements imposed on industry, which are becoming more and more exacting.

“Useful organic molecule” means a compound resulting from the degradation/depolymerization of lignin or of a derivative thereof which is deemed sufficiently interesting to require its production/utilization/isolation from the crude reaction mixture.

“Lignin or derivatives thereof” means lignin as generally defined in this technical field, but also any other lignin derivative (for example lignosulfonates), obtained from all known or unknown sources of biomass (for example those obtained from wood of conifers, or from black liquor from conifers), and in all its forms (for example before or after pretreatment).

According to a first aspect, the invention therefore relates to a method for the depolymerization of lignin or a derivative thereof, comprising a step of heating lignin or a derivative thereof in the presence of a hydroxide of general formula M(OH)n or a mixture of hydroxides M(OH)n; in this formula M is a metal of the alkali or alkaline-earth family and n is equal to 1 or 2, in which the weight ratio between said hydroxide or mixture of hydroxides and lignin or a derivative thereof is preferably between about 0.5 and about 20.

Even more preferably, the weight ratio between said hydroxide or mixture of hydroxides and lignin or a derivative thereof is between about 0.5 and about 10.

Of course, this weight ratio can be greater than 20, i.e. with the lignin in very dilute conditions, without departing from the essence of the present invention but simply to the detriment, for example, of the costs associated with the implementation of the method.

The step of heating lignin or a derivative thereof is advantageously carried out at a temperature between the melting point of said hydroxide or mixture of hydroxides and a temperature equal to said melting point plus about 150 degrees Celsius, preferably equal to said melting point plus about 100 degrees Celsius.

The method of depolymerization can also include an additional step of treatment of the product obtained by said depolymerization.

Preferably, said hydroxide is selected from lithium hydroxide (LiOH), sodium hydroxide (NaOH), potassium hydroxide (KOH) or calcium hydroxide (Ca(OH)₂), NaOH and KOH being particularly preferred.

When a mixture of hydroxides is used, it advantageously comprises NaOH. It is possible to use a mixture of two hydroxides; it is then called a binary mixture. The molar ratio between the hydroxides of the binary mixture is generally between about 5/95 and about 95/5, for example between about 20/80 and about 80/20, between about 30/70 and about 70/30, or between about 40/60 and about 60/40. Advantageously, the binary mixture of hydroxides comprises NaOH and preferably consists of NaOH and KOH.

It is also possible to use a mixture of three hydroxides; it is then called a ternary mixture. Each hydroxide is present in the ternary mixture in a molar quantity that can represent from about 1% to about 50% of the mixture.

Preferably, said mixture of hydroxides is a eutectic mixture.

“Eutectic mixture” means a mixture of at least two products in definite proportions possessing physicochemical characteristics essentially identical to those of a single product or “pure substance”. The eutectic mixture can be binary and can therefore consist of a mixture of two products but can also comprise more than two products. In the present case, a mixture of so-called “eutectic” hydroxides will have a melting point essentially equal to its solidification temperature.

Advantageously, said hydroxide or mixture of hydroxides has a melting point of less than 300° C., preferably less than 250° C., more preferably less than 200° C. It should be noted that in the case of a binary eutectic mixture comprising sodium hydroxide and potassium hydroxide, the melting point of said mixture is approximately equal to 172° C. This eutectic mixture is obtained by mixing the two aforementioned hydroxides in a molar ratio substantially equal to 1:1.

It is also important to note that ranges of temperature values are not limited to the aforementioned values. Any melting point of said hydroxide or mixture of hydroxides allowing the application of the method according to the invention in conditions satisfying one or more of the aforementioned advantages must be regarded as forming an integral part of the invention. In fact, the lower the temperature at which the method according to the invention is carried out, the greater will be the energy saving and therefore the more the method will be economically viable.

The method of the invention can therefore be carried out at a temperature not exceeding about 300° C., preferably between about 180° C. and about 250° C., more preferably between about 200° C. and about 250° C.

Advantageously, the depolymerization reaction is carried out at atmospheric pressure. However, the reaction can also be carried out under a higher pressure to allow management of the gases present in the mixture or under a lower pressure so as to facilitate the extraction of one or more compounds of interest.

The reaction is generally carried out for a time comprised between about 1 h and about 20 h, preferably between about 1 h and about 5 h.

As a guide, the depolymerization reaction according to the method of the invention is preferably carried out at a reaction temperature of about 200° C., for a time of about 2 h.

According to an essential aspect of the invention, no transition metal is used during application of the depolymerization reaction according to the method of the invention.

The depolymerization reaction can be carried out with or without stirring. A person skilled in the art will adjust the stirring speed as necessary, depending on the reactor used, the nature of the starting products (lignin or derivatives thereof and hydroxides), and the volume to be stirred.

The method according to the invention may comprise, besides the step of heating lignin or a derivative thereof as described above, in which the hydroxide or the mixture of hydroxides acts both as a solvent and as a depolymerization reagent, one or more other steps such as:
a preliminary step of heating the hydroxide or the mixture of hydroxides in a suitable vessel until liquefaction occurs,
a step of treatment of the product of depolymerization, preferably comprising an acidification step followed by an extraction step.

The method may also comprise a step of separation and/or of purification of the products obtained in the depolymerization step.

In the method according to the invention it is possible to use lignin or one or more derivatives thereof of diverse origin. Amongst these products, it may be mentioned lignin obtained from wood of conifers, from pine chips or from cane from Provence, lignin from annual plants, black liquor from conifers, or the lignosulfonates, which are preferably used in the present method. Of course, a person skilled in the art will understand that the method according to the invention can be applied to any crude, purified or pretreated mixture, containing lignin or one or more derivatives thereof.

As mentioned above, against all expectations, it was found, quite surprisingly, that the method of depolymerization according to the invention can be carried out with a great variety of substrates containing lignin but without particularly affecting the quality of the mixture of products of continuous depolymerization (or in other words the ease of purification of the mixture of products of depolymerization obtained and the degree of conversion of the depolymerization reaction).

Moreover, it should be noted that the organic compounds obtained by the method according to the invention are generally aromatic and essentially belongs to the family of phenols, benzoic acids or anisoles. Amongst the organic compounds that can be obtained, notably in the form of a mixture, it may be mentioned for example guaiacol, ortho-methoxycresol, homovanillic acid, hydroferulic acid, vanillic acid, veratric acid and protocatechuic acid.

Thus, according to another aspect, the invention relates to a product of depolymerization obtained by the method of depolymerization according to the invention. It is notably possible to obtain a product of depolymerization essentially comprising at least one compound selected from guaiacol, ortho-methoxycresol, homovanillic acid, hydroferulic acid, vanillic acid, veratric acid and protocatechuic acid, for example a mixture of two, three, four, five or six of these compounds.

The organic products isolated from the mixture of products of depolymerization of lignin obtained according to the invention can be used in many industries for numerous applications.

Amongst these industries we may mention the cosmetics industry, the food industry, the pharmaceutical industry and the manufacture of polymers.

The invention will be better understood from the examples given below for illustration purpose only.

### EXAMPLE 1

### Reaction of Depolvmerization According to the Invention

In this example, a commercial lignin produced from conifer wood and marketed by the company Aldrich was used. This lignin is in the form of a fine brown powder.

The depolymerization reaction was carried out in an ionothermal or “molten salt” medium in which a sodium hydroxide/potassium hydroxide (NaOH/KOH) eutectic with melting point of 172° C. was used.

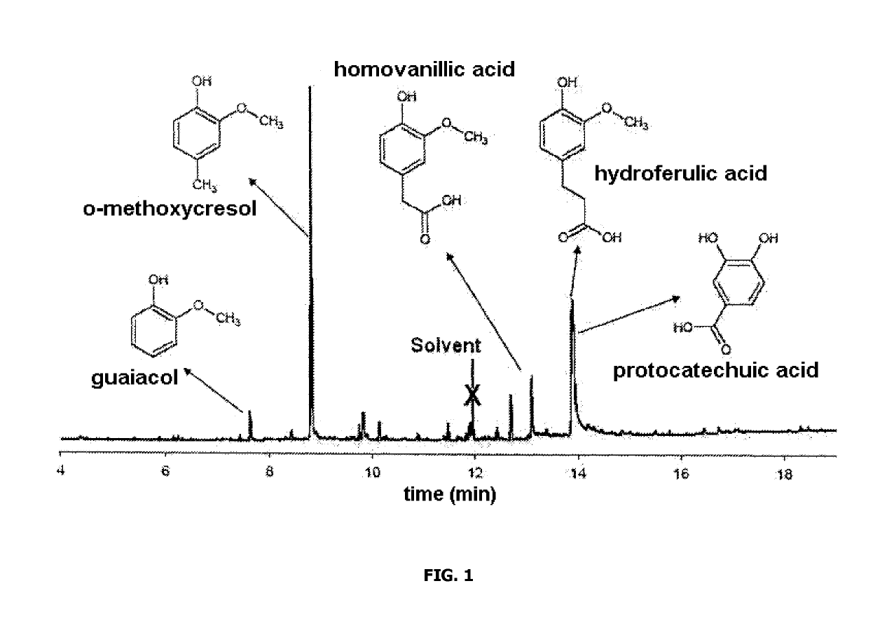

The protocol as well as the operating conditions used in this particular example are described as follows:
10 g of a NaOH/KOH mixture (4.28 g/5.72 g equivalent to a molar ratio of 1:1), ground and mixed beforehand, was placed in a Teflon reactor with a capacity of about 30 mL. The crucible was heated at 200° C. in a muffle furnace for one hour until liquefaction of the mixture occurred.
1 g of commercial lignin produced from conifer wood (Aldrich) was then added hot in the molten salt with stirring. The mixture was then heated for 2 hours without stirring.
At the end of the reaction time, the crucible was removed from the furnace and was left to cool to room temperature. All of the reagents were then dissolved in 50 mL of distilled water and acidified with 37% concentrated hydrochloric acid until a precipitate formed (pH<2).
Then the organic phase was extracted in a separating funnel with 3×25 mL of diethyl ether. The organic phases were combined, dried over anhydrous magnesium sulfate MgSO₄, then filtered through Celite.
The organic solvent was removed using a rotary evaporator and 110 mg of mixture was obtained.
This mixture was then taken up in 5 mL of a solution of toluene (0.05M, as internal standard) in diethyl ether and was analyzed by gas chromatography coupled to a mass spectrometer (the corresponding chromatogram is presented in FIG. 1 including attribution of the main observed products).

After analysis, the mixture obtained was also distilled in a bubbling furnace at atmospheric pressure, giving 20 mg of a fraction obtained between 220 and 250° C., consisting of pure o-methoxycresol.

### EXAMPLES 2 to 21

### Variation of the Reaction Conditions

In the following examples, the protocol used in example 1 was reproduced but with variation of certain parameters, such as the nature of the hydroxides used, the water content of these hydroxides, the time and temperature of the depolymerization reaction.

The various reaction products were treated in the same way as was described in example 1. The reaction conditions and the results obtained are presented in Table 1 below.

TABLE 1

| | Mixture of | Mass of | | Reaction | mass of | | Amount of o- |
|---|---|---|---|---|---|---|---|
| | hydroxides | hydroxides | | time | lignin | Water | methoxycresol |
| Ex. | (mol/mol) | (g) | Temp. (° C.) | (h) | (mg) | content^{a} | formed (mg)^{b} |
| 2 | NaOH/KOH | 5 | 200 | 0.5 | 250 | Native | 0.44 |
| | 1/1 | | | | | | |
| 3 | NaOH/KOH | 5 | 200 | 1 | 250 | Native | 0.58 |
| | 1/1 | | | | | | |
| 4 | NaOH/KOH | 5 | 200 | 2 | 250 | Native | 0.77 |
| | 1/1 | | | | | | |
| 5 | NaOH/KOH | 5 | 200 | 4 | 250 | Native | 0.62 |
| | 1/1 | | | | | | |
| 6 | NaOH/KOH | 10 | 200 | 20 | 500 | Native | 0.00^{c} |
| | 1/1 | | | | | | |
| 7 | NaOH/KOH | 5 | 200 | 0.5 | 250 | Saturated | 0.52 |
| | 1/1 | | | | | | |
| 8 | NaOH/KOH | 5 | 200 | 1 | 250 | Saturated | 0.66 |
| | 1/1 | | | | | | |
| 9 | NaOH/KOH | 5 | 200 | 2 | 250 | Saturated | 0.60 |
| | 1/1 | | | | | | |
| 10 | NaOH/KOH | 5 | 200 | 4 | 250 | Saturated | 0.49 |
| | 1/1 | | | | | | |
| 11 | NaOH/KOH | 5 | 200 | 0.5 | 250 | Dry | 0.72 |
| | 1/1 | | | | | | |
| 12 | NaOH/KOH | 5 | 200 | 1 | 250 | Dry | 0.62 |
| | 1/1 | | | | | | |
| 13 | NaOH/KOH | 5 | 200 | 2 | 250 | Dry | 0.84 |
| | 1/1 | | | | | | |
| 14 | NaOH/KOH | 5 | 200 | 4 | 250 | Dry | 0.83 |
| | 1/1 | | | | | | |
| 15 | NaOH/KOH | 10 | 200 | 2 | 500 | Dry | 1.88 |
| | 1/1 | | | | | | |
| 16 | NaOH/KOH | 10 | 200 | 2 | 1000 | Dry | 5.18 |
| | 1/1 | | | | | | |
| 17 | NaOH/KOH | 10 | 220 | 2 | 500 | Native | 1.43 |
| | 1/1 | | | | | | |
| 18 | NaOH/KOH | 10 | 250 | 2 | 500 | Native | 0.00^{c} |
| | 38/62 | | | | | | |
| 19 | NaOH/KOH | 10 | 300 | 2 | 500 | Native | 0.00^{c} |
| | 93/7 | | | | | | |
| 20 | NaOH/KOH | 10 | 200 | 2 | 500 | Native | 1.35 |
| | 1/1 + 5% | | | | | | |
| | Ca(OH)₂^{d} | | | | | | |
| 21 | LiOH/NaOH | 10 | 200 | 2 | 500 | Saturated | 0.57 |
| | 1/1 | | | | | | |

^{a}Measurements of the water-content of the hydroxides as a function of time elapsed at 200° C. were also carried out. “Native” means the mixture of hydroxides prepared on the bench without particular precautions and containing less than 3 wt % of water; “dry” means a mixture of hydroxides prepared from dry hydroxides in a glove box; “saturated” means a mixture of hydroxides containing about 8 wt % of water corresponding to a hydroxide whose water content varies very little with time at 200° C.
^{b}Masses determined by GC-MS using toluene as internal reference.
^{c}The main product detected is protocatechuic acid
^{d}The percentage by weight of Ca(OH)₂ relative to the NaOH/KOH mixture

It should be noted that the GC-MS chromatograms obtained for examples 2 to 21 indicate qualitatively that the same depolymerization products were formed as in the conditions used in example 1.

As shown in Table 1, the mass of ortho-methoxycresol was determined for each example, since it was realized that the amount of ortho-methoxycresol obtained is a good indicator of the progress of depolymerization. The choice of ortho-methoxycresol is therefore purely arbitrary and makes it possible, among other things, to follow the development of the reaction more easily.

It should also be noted that the amounts measured are less than those obtained by distillation in example 1. The masses indicated, being extrapolated from the GC-MS results, therefore seem to be less than the “true” masses that would result from purification by distillation.

Based on the results obtained, summarized in Table 1, it may be noted that the nature of the hydroxides used as well as their water content seems to have only a slight influence on the amount of o-methoxycresol formed.

Nevertheless, the experimental conditions used in example 13 prove to be particularly favorable for obtaining a significant amount of ortho-methoxycresol and therefore could be regarded as the experimental conditions offering the best yield in the depolymerization reaction.

Finally, it should be noted that “scaling-up” is favorable to the depolymerization reaction according to the invention since the relative yield of o-methoxycresol doubles on passing from 250 mg to 1 g of starting product.

Finally, it may be stated that for longer reaction times, protocatechuic acid is obtained very selectively at a very good yield. In fact in example 6, 330 mg of organosoluble product was isolated, most of which is protocatechuic acid.

### EXAMPLES 22 to 26

### Variation of the Lignin-Containing Source

In the following examples, the protocol used in example 1 was followed, with different types of lignocellulosic biomasses.

In general, each example was carried out with 10 g of a 1/1 “native” NaOH/KOH mixture and 500 mg of biomass containing lignin (substrate), held at a temperature of 200° C. for 2 h. Table 2 below summarizes the results obtained with the different biomasses tested.

TABLE 2

| | | Amount of | |
|---|---|---|---|
| | | o-methoxycresol | Corresponding |
| Ex. | Substrate | formed (mg)^{a} | figure |
| 22 | Pine chips | 2.09 | 2 |
| 23 | Cane from | n.d.^{b} | 3 |
| | Provence | | |
| 24 | Lignin from | 2.04 | 4 |
| | annual | | |
| | plants | | |
| 25 | Black liquor | 0.12^{c} | 5 |
| | from | | |
| | conifers | | |
| 26 | Ligno- | 1.00 | 6 |
| | sulfonates | | |
| | (Booregaard) | | |

^{a}Masses determined by GC-MS using toluene as internal reference.
^{b}p-Hydroxybenzoic acid is mostly obtained.
^{c}It should be noted that the black liquor used contains a certain amount of water and therefore the yield by weight cannot be determined in this case.

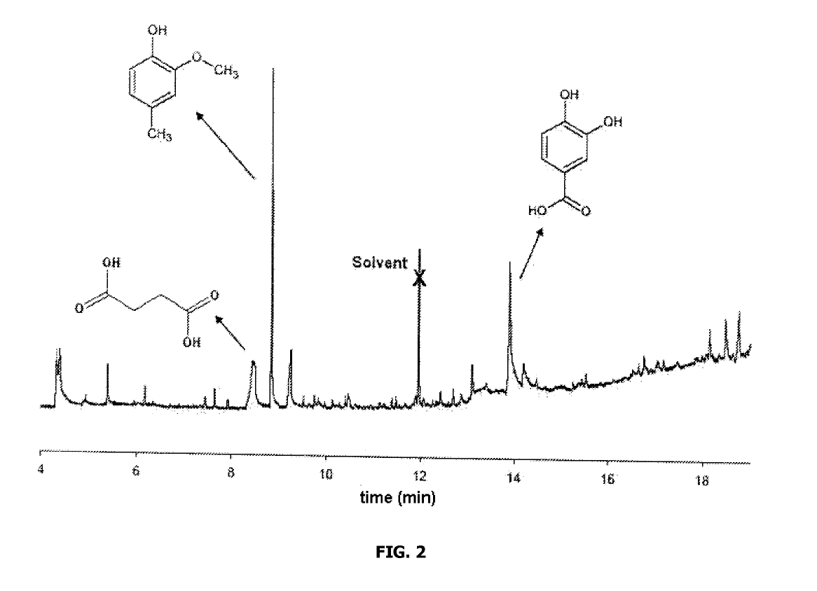

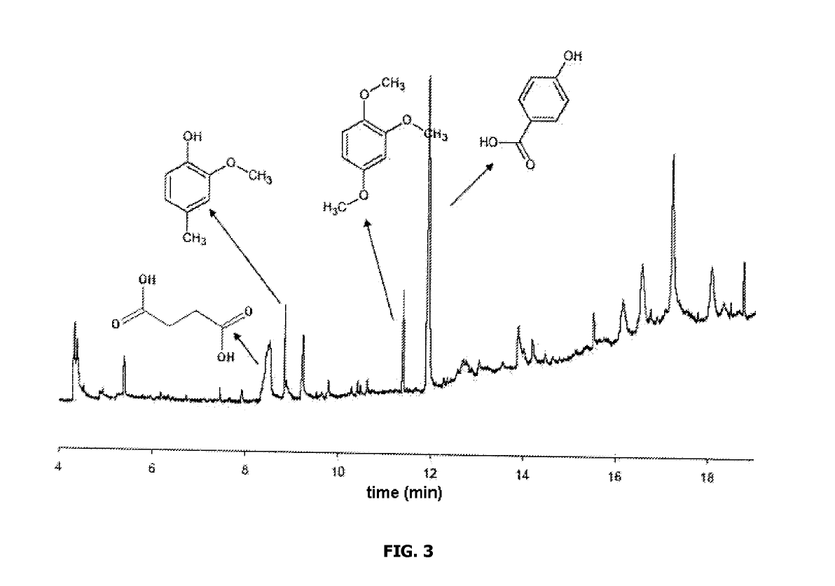

It can be seen that the yields and the purity of the products obtained are better when the crude biomass is used (examples 22 and 23, FIGS. 2 and 3 respectively).

1. A method for the depolymerization of lignin or a derivative thereof, comprising a step of heating lignin or a derivative thereof in the presence of a hydroxide of general formula M(OH)n or a mixture of hydroxides M(OH)n wherein, in the formula, M is an alkali or alkaline-earth metal and n is equal to 1 or 2,
in which the weight ratio between said hydroxide or mixture of hydroxides and lignin or a derivative thereof is comprised between about 0.5 and about 20, and
in which the heating step is carried out at a temperature comprised between the melting point of said hydroxide or mixture of hydroxides and a temperature equal to said melting point plus 150 degrees Celsius.

2. The method as claimed in claim 1, in which the heating step is carried out at a temperature comprised between the melting point of said hydroxide or mixture of hydroxides and a temperature equal to said melting point plus 100 degrees Celsius.

3. The method as claimed in claim 1, comprising an additional treatment step of the product of said depolymerization.

4. The method as claimed in claim 1, in which said hydroxide is selected from the group consisting of LiOH, NaOH, KOH and Ca(OH)₂.

5. The method as claimed in claim 1, in which heating is carried out in the presence of a mixture of hydroxides comprising NaOH.

6. The method as claimed in claim 1, in which said mixture of hydroxides is a mixture consisting of NaOH and KOH.

7. The method as claimed in claim 1, in which said mixture of hydroxides is an eutectic mixture.

8. The method as claimed in claim 1, in which said hydroxide or mixture of hydroxides has a melting point of less than 300° C.

9. The method as claimed in claim 1, in which the depolymerization reaction is carried out under atmospheric pressure.

10. The method as claimed in claim 1, in which said lignin or derivative thereof is selected from the group consisting of lignin obtained from wood of conifers, pine chips or cane from Provence, lignin from annual plants, black liquor from conifers, and lignosulfonates.

11. A depolymerization product obtainable by the method as claimed in claim 1.

12. The method as claimed in claim 1, in which the weight ratio between said hydroxide or mixture of hydroxides and lignin or a derivative thereof is comprised between about 0.5 and about 10.

13. The method as claimed in claim 2, in which said mixture of hydroxides is a mixture consisting of NaOH and KOH.

14. The method as claimed in claim 2, in which said mixture of hydroxides is an eutectic mixture.

15. The method as claimed in claim 6, in which said mixture of hydroxides is an eutectic mixture.

16. The method as claimed in claim 1, in which said hydroxide or mixture of hydroxides has a melting point of less than 250° C.

17. The method as claimed in claim 1, in which said hydroxide or mixture of hydroxides has a melting point of less than 200° C.

18. The method as claimed in claim 2, in which the depolymerization reaction is carried out under atmospheric pressure.

19. The method as claimed in claim 6, in which the depolymerization reaction is carried out under atmospheric pressure.

20. The method as claimed in claim 15, in which the depolymerization reaction is carried out under atmospheric pressure.
